(19) United States
(12) Patent Application Publication
(10) Pub. No.: US 2023/0172434 A1
(43) Pub. Date: Jun. 8, 2023

(54) ENDOSCOPE

(71) Applicant: FUJIFILM Corporation, Tokyo (JP)

(72) Inventors: Shin IWATA, Kanagawa (JP); Nobuyuki TORISAWA, Kanagawa (JP)

(73) Assignee: FUJIFILM Corporation, Tokyo (JP)

(21) Appl. No.: 18/162,476

(22) Filed: Jan. 31, 2023

Related U.S. Application Data
(63) Continuation of application No. PCT/JP2021/030827, filed on Aug. 23, 2021.

(30) Foreign Application Priority Data
Aug. 28, 2020 (JP) 2020-144324
Jun. 10, 2021 (JP) 2021-097243

(51) Int. Cl. A61B 1/00; A61B 1/05

(52) U.S. Cl. CPC A61B 1/00121; A61B 1/00135; A61B 1/05

(57) ABSTRACT

An endoscope includes: a protection sheath that forms an insertion unit; a pipe-like case that is connected to a proximal end side of the protection sheath; a distal end optical system that is provided at a distal end of the protection sheath and that defines a distal end side of a sealed space formed in the protection sheath and the case; a partition wall that is provided in the case, is perpendicular to an insertion axis of the insertion unit, and defines a proximal end side of the sealed space; a shaft member that is inserted into the protection sheath and that is rotatable relative to the protection sheath in a direction around the insertion axis; an image pickup unit that is provided at a distal end of the shaft member and that picks up an image of light passing through the distal end optical system; and a magnet coupling which includes a first magnet provided in the sealed space and a second magnet provided outside the sealed space with the partition wall interposed therebetween and of which the first magnet is connected to a proximal end side of the shaft member. The magnet coupling and the case are rotatable relative to each other in the direction around the axis.

### CROSS-REFERENCE TO RELATED APPLICATIONS

The present application is a Continuation of PCT International Application No. PCT/JP2021/030827 filed on Aug. 23, 2021 claiming priorities under 35 U.S.C § 119(a) to Japanese Patent Application No. 2020-144324 filed on Aug. 28, 2020 and Japanese Patent Application No. 2021-097243 filed on Jun. 10, 2021. Each of the above applications is hereby expressly incorporated by reference, in its entirety, into the present application.

### BACKGROUND OF THE INVENTION

### 1. Field of the Invention

The present invention relates to an endoscope including an insertion unit.

### 2. Description of the Related Art

A rigid endoscope is known as an endoscope used for endoscopic surgery or the like. Further, an oblique-viewing endoscope of which an oblique front side with respect to an insertion axis (also referred to as a longitudinal axis) of an insertion unit corresponds to a visual field direction is known as this rigid endoscope. The oblique-viewing endoscope comprises an insertion unit that is to be inserted into an object to be examined and an operation unit that is connected to a proximal end portion of the insertion unit. This insertion unit includes, for example, a protection sheath in which a distal end optical system (oblique-viewing optical system) is provided at a distal end portion, and an inner sheath which is inserted into the protection sheath and in which an image pickup unit is provided at a distal end portion (see JP2015-507497A and US2019/0117048A). A sealed space is formed in the protection sheath, and the inner sheath is disposed in the sealed space.

As disclosed in US2019/0117048A, the operation unit comprises a cylindrical operation unit body, and a cylindrical operation ring that is held on an outer peripheral surface of a distal end portion of the operation unit body to be relatively rotatable. A proximal end portion of the inner sheath is inserted into the operation unit body. A proximal end portion of the protection sheath is connected to the operation ring. Accordingly, in a case where the operation ring is rotated in a direction around the insertion axis of the insertion unit, the protection sheath and the distal end optical system can be rotated in the same direction. As a result, the observation direction (visual field direction) of the oblique-viewing endoscope can be rotated.

Since an observation image to be observed by a practitioner is also rotated in a screen of a monitor in a case where the inner sheath (image pickup unit) is rotated together with the protection sheath at this time, there is a concern that it may be difficult for a practitioner to observe the observation image.

Accordingly, the oblique-viewing endoscope disclosed in US2019/0117048A is provided with a magnet coupling. The magnet coupling comprises a plurality of magnets that are concentrically arranged about the insertion axis of the insertion unit, more specifically, a plurality of inner magnets that are provided on an outer peripheral surface of the inner sheath in a circumferential direction thereof and a plurality of outer ring magnets that are provided on an inner peripheral surface of the operation unit body in the circumferential direction thereof. Torque [stop (holding) torque] can be transmitted to the inner sheath from the operation unit body in a contactless manner by this magnet coupling. As a result, even in a case where the protection sheath is rotated using the operation ring, the posture of the inner sheath in the direction around the axis can be maintained.

### SUMMARY OF THE INVENTION

In a case where the magnet coupling disclosed in US2019/0117048A is provided in the operation unit, the inner magnets and the outer ring magnets need to be concentrically arranged about the insertion axis, that is, the inner magnets are provided on the outer peripheral surface of the inner sheath and the outer ring magnets need to be provided on the inner peripheral surface of the operation unit body. As a result, there is a problem in that the diameter of the operation unit is increased.

The present invention has been made in consideration of such circumstances, and an object of the present invention is to provide an endoscope in which an increase in diameter of an operation part can be prevented.

In order to achieve the object of the present invention, an endoscope according to an aspect of the present invention comprises a protection sheath that forms an insertion unit, a pipe-like case that is connected to a proximal end side of the protection sheath, a distal end optical system that is provided at a distal end of the protection sheath and that defines a distal end side of a sealed space formed in the protection sheath and the case, a partition wall that is provided in the case, is perpendicular to an insertion axis of the insertion unit, and defines a proximal end side of the sealed space, a shaft member that is inserted into the protection sheath and that is rotatable relative to the protection sheath in a direction around the insertion axis, an image pickup unit that is provided at a distal end of the shaft member and that picks up an image of light passing through the distal end optical system, and a magnet coupling which includes a first magnet provided in the sealed space and a second magnet provided outside the sealed space with the partition wall interposed therebetween and of which the first magnet is connected to a proximal end side of the shaft member. The magnet coupling and the case are rotatable relative to each other in the direction around the axis.

According to this endoscope, the magnet coupling acting in a thrust direction of the insertion axis can be used to maintain the postures of the shaft member and the image pickup unit, which are provided in the sealed space, in the direction around the axis with respect to the protection sheath and the distal end optical system or to rotate the shaft member and the image pickup unit in the direction around the axis.

According to another aspect of the present invention, the endoscope further comprises a signal line that is connected to the image pickup unit, and each of the first magnet and the second magnet is formed in a shape of a disk perpendicular to the insertion axis and includes an insertion hole into which the signal line is to be inserted. Accordingly, the signal line can be inserted into the first magnet and the second magnet.

According to another aspect of the present invention, the endoscope further comprises a signal line that is connected to the image pickup unit, each of the first magnet and the second magnet includes a magnet holding part that includes an insertion hole into which the signal line is to be inserted and is formed in an annular shape as viewed from the partition wall, and a plurality of individual magnets that are provided on the magnet holding part at intervals and that have magnetic poles in an axial direction of the insertion axis, and the magnet holding part of the first magnet is connected to the proximal end side of the shaft member. Accordingly, both an improvement in transmission torque and a reduction in the amount of slip can be achieved in the magnet coupling.

According to another aspect of the present invention, in the endoscope, the plurality of individual magnets are provided on the magnet holding part in the direction around the axis.

According to another aspect of the present invention, in the endoscope, the plurality of individual magnets are provided on the magnet holding part at regular intervals in the direction around the axis.

According to another aspect of the present invention, in the endoscope, magnetic poles of one of the individual magnets adjacent to each other in the direction around the axis are reversed with respect to magnetic poles of the other of the individual magnets.

According to another aspect of the present invention, in the endoscope, the plurality of individual magnets are eccentric to an outer peripheral side of the magnet holding part in a case where the magnet holding part is viewed from the partition wall.

According to another aspect of the present invention, in the endoscope, the individual magnet has a shape extending in a direction parallel to the insertion axis.

According to another aspect of the present invention, in the endoscope, the signal line includes a first signal line disposed in the sealed space and a second signal line disposed outside the sealed space, and the endoscope further comprises an airtight connector that is provided in the partition wall and that connects the first signal line to the second signal line. Accordingly, image pickup signals of the image pickup unit can be output from the inside of the sealed space to outside the sealed space.

According to another aspect of the present invention, in the endoscope, the shaft member is an inner sheath into which the signal line is inserted, the endoscope further comprises a proximal end optical system that is provided at a distal end of the inner sheath and that guides light, which passes through the distal end optical system, to the image pickup unit, the image pickup unit includes an image pickup element that picks up an image of light incident through the proximal end optical system and that outputs an image pickup signal to the signal line, and the distal end optical system, the proximal end optical system, and the image pickup element are rotatable relative to each other in the direction around the axis. Accordingly, the postures of the proximal end optical system and the image pickup element, which are provided in the sealed space, in the direction around the axis can be maintained with respect to the protection sheath and the distal end optical system, or the proximal end optical system and the image pickup element can be rotated in the direction around the axis.

According to another aspect of the present invention, in the endoscope, the proximal end optical system includes a proximal end lens barrel that is connected to the distal end of the inner sheath, and an image pickup element-mounting part which is connected to a proximal end side of the proximal end lens barrel and on which the image pickup element is mounted. Accordingly, the proximal end lens barrel and the image pickup element can be integrally rotated in the direction around the axis.

According to another aspect of the present invention, in the endoscope, the distal end optical system includes a distal end portion body and a distal end lens barrel fixed to the distal end portion body.

According to another aspect of the present invention, in the endoscope, the shaft member is an inner sheath, the endoscope further comprises a proximal end optical system that is provided at a distal end of the inner sheath and that guides light, which passes through the distal end optical system, to the image pickup unit, the proximal end optical system includes a proximal end lens barrel that is connected to the distal end of the inner sheath, and one of the distal end lens barrel and the proximal end lens barrel is fitted to the other of the distal end lens barrel and the proximal end lens barrel to be relatively rotatable in the direction around the axis.

According to another aspect of the present invention, the endoscope further comprises a first bearing receiving member that is fixed to the first magnet in the sealed space of the case, and a first bearing that is fixed to the first bearing receiving member and that is inscribed in the case, and the magnet coupling and the case are rotatable relative to each other in the direction around the axis via the first bearing. Accordingly, the magnet coupling and the case are rotatable relative to each other in the direction around the axis.

According to another aspect of the present invention, in the endoscope, the proximal end side of the shaft member is connected to the first bearing receiving member. Accordingly, the postures of the shaft member and the image pickup unit, which are provided in the sealed space, in the direction around the axis can be maintained with respect to the protection sheath and the distal end optical system, or the shaft member and the image pickup unit can be rotated in the direction around the axis.

According to another aspect of the present invention, the endoscope further comprises an outer pipe into which the protection sheath is inserted, and a light guide that is disposed in a space between the outer pipe and the protection sheath.

According to another aspect of the present invention, in the endoscope, the distal end optical system is fixed to a distal end side of the outer pipe, and the endoscope further comprises an operation part that is connected to a proximal end side of the outer pipe and that houses the case. In a case where a rotational force for rotating the operation part in the direction around the axis is applied to the operation part, the rotational force is transmitted to the protection sheath and to the case via the outer pipe and the distal end optical system. Accordingly, the distal end optical system, the protection sheath, and the case can be rotated in the direction around the axis.

According to another aspect of the present invention, the endoscope further comprises a tubular portion that is provided on a proximal end side of the case, a second bearing receiving member that is fixed to the second magnet outside the sealed space, a second bearing that is fixed to the second bearing receiving member and that is inscribed in the tubular portion, a pipe-like extending part that is provided on a proximal end side of the operation part and that is rotatable relative to the operation part in the direction around the axis, and an extension part that is provided in the extending part and that connects the extending part to the second bearing receiving member. Accordingly, torque output from the extending part and from the extension part can be transmitted to the shaft member and to the image pickup unit via the magnet coupling.

According to another aspect of the present invention, in the endoscope, the shaft member is an inner sheath, the endoscope further comprises a proximal end optical system that is provided at a distal end of the inner sheath and that guides light, which passes through the distal end optical system, to the image pickup unit, the image pickup unit includes an image pickup element that picks up an image of light incident through the proximal end optical system, and the proximal end optical system includes a proximal end lens barrel that is fixed to the distal end of the inner sheath, a first refractive optical element that is connected to the image pickup element and that refracts light, which is incident from the proximal end optical system, toward the image pickup element, and a holder that holds the first refractive optical element on a proximal end side of the proximal end lens barrel.

According to another aspect of the present invention, in the endoscope, the distal end optical system includes a second refractive optical element that refracts light, which is incident in a direction inclined with respect to the insertion axis, parallel to the insertion axis. Accordingly, an oblique front side with respect to the insertion axis can be observed.

According to another aspect of the present invention, in the endoscope, the distal end optical system includes a distal end portion body and a distal end lens barrel fixed to the distal end portion body, and the distal end lens barrel houses the second refractive optical element.

According to the present invention, an increase in diameter of the operation part can be prevented.

### BRIEF DESCRIPTION OF THE DRAWINGS

FIG. 1 is a diagram showing a configuration of an endoscope system that comprises an oblique-viewing endoscope.

FIG. 2 is an enlarged cross-sectional view of a distal end portion of an insertion unit.

FIG. 3 is a cross-sectional view of an operation part.

FIG. 4 is a cross-sectional view of a protection sheath and a case.

FIG. 5 is an enlarged cross-sectional view of the case and a tubular portion.

FIG. 6 is a front view of a first magnet and a second magnet as viewed from a partition wall side.

FIG. 7 is a side view of the first magnet and the second magnet.

FIG. 8 is an enlarged cross-sectional view of a case and a tubular portion that are provided in an operation part of an oblique-viewing endoscope according to a second embodiment.

FIG. 9 shows enlarged cross-sectional views of the first magnet and the second magnet shown in FIG. 8, and enlarged front views of the first magnet and the second magnet as viewed from the partition wall side, respectively.

FIG. 10 is a front view of a first magnet and a second magnet of Modification Example 1 of the second embodiment as viewed from a partition wall side, respectively.

FIG. 11 is a front view of a first magnet and a second magnet of Modification Example 2 of the second embodiment as viewed from a partition wall side, respectively.

FIG. 12 is a front view of a first magnet and a second magnet of Modification Example 3 of the second embodiment as viewed from a partition wall side, respectively.

FIG. 13 is a front view of a first magnet and a second magnet of Modification Example 4 of the second embodiment as viewed from a partition wall side, respectively.

FIG. 14 is a front view of a first magnet and a second magnet of Modification Example 5 of the second embodiment as viewed from a partition wall side, respectively.

### DESCRIPTION OF THE PREFERRED EMBODIMENTS

[Endoscope System]

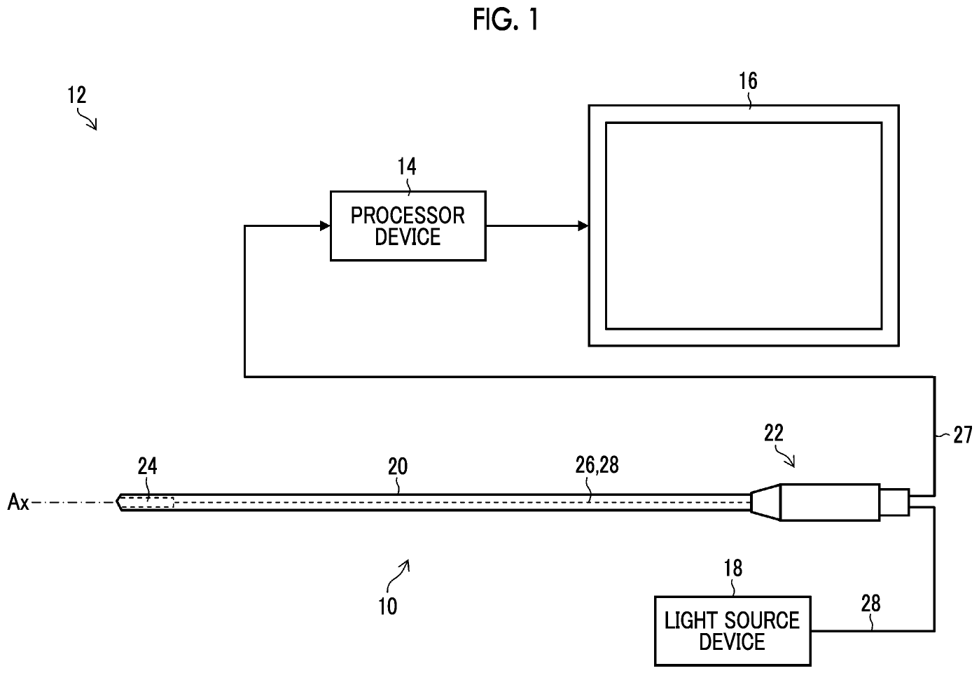

FIG. 1 is a diagram showing the configuration of an endoscope system 12 that comprises an oblique-viewing endoscope 10. As shown in FIG. 1, the endoscope system 12 comprises the oblique-viewing endoscope 10 according to a first embodiment corresponding to an endoscope of the present invention, a processor device 14, a monitor 16, and a light source device 18.

[Oblique-Viewing Endoscope According to First Embodiment]

The oblique-viewing endoscope 10 is a so-called rigid endoscope and comprises an insertion unit 20 and an operation part 22. The insertion unit 20 is formed substantially in the shape of a pipe (in a substantially tubular shape), and is inserted into a patient's body. The insertion unit 20 has a distal end, a proximal end, and an insertion axis Ax. A camera unit 24 to be described later is provided at a distal end portion of the insertion unit 20. Further, a first signal line 26 (signal cable) and a light guide 28 (optical fiber cable) are inserted into the insertion unit 20.

The first signal line 26 connects the camera unit 24 to be described later to the processor device 14 to be described later together with a second signal line 27 to be described later. A distal end portion of the first signal line 26 is connected to the camera unit 24, and a proximal end portion of the first signal line 26 is connected to the second signal line 27 in the operation part 22. Accordingly, the first signal line 26 and the second signal line 27 correspond to a signal line of the present invention. A distal end portion (light emitting end surface) of the light guide 28 is provided on a distal end surface of the insertion unit 20, and a proximal end portion (light incident end surface) thereof is connected to the light source device 18.

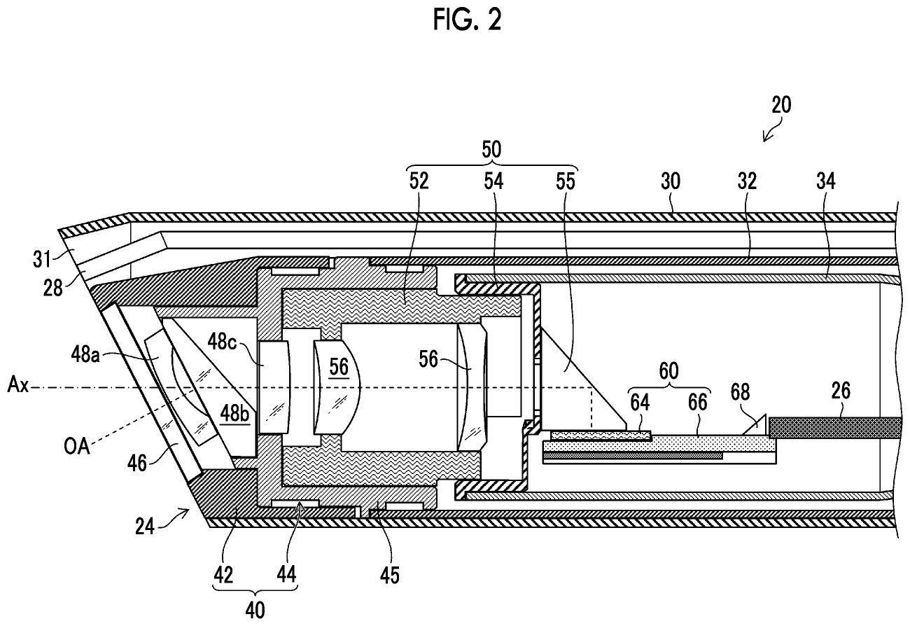

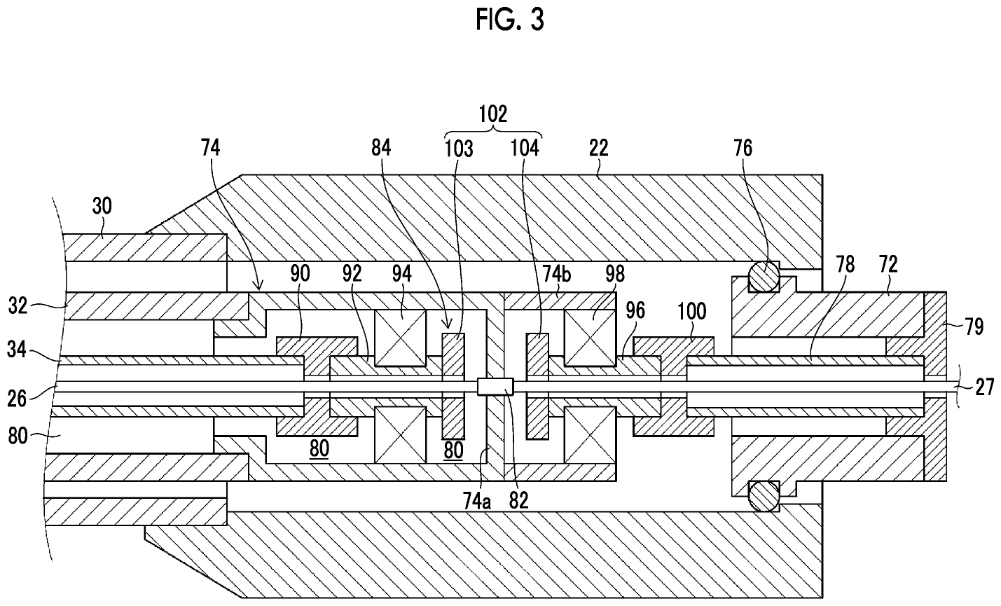

The operation part 22 receives a rotating operation for rotating a visual field direction of the oblique-viewing endoscope 10 (an observation direction, see an optical axis OA shown in FIG. 2) in a direction around the insertion axis Ax (a circumferential direction of the insertion unit 20 and the operation part 22). Further, as described in detail later, the operation part 22 includes an airtight space and a non-airtight space therein, and the proximal end portion of the first signal line 26 and a distal end portion of the second signal line 27 are connected to each other at a boundary between both of the spaces (see FIG. 3). A proximal end portion of the second signal line 27 is connected to the processor device 14. Accordingly, the camera unit 24 and the processor device 14 are electrically connected to each other via the first signal line 26 and the second signal line 27.

The processor device 14 generates an observation image (video) of the inside of the patient's body on the basis of image pickup signals, which are input from the camera unit 24 through the first signal line 26 and the second signal line 27, and causes the monitor 16 to display this observation image.

The light source device 18 supplies illumination light to the light guide 28. Accordingly, illumination light is emitted from the light emitting end surface of the distal end portion of the light guide 28 provided on the distal end surface of the insertion unit 20.

FIG. 2 is an enlarged cross-sectional view of the distal end portion of the insertion unit 20. As shown in FIG. 2, the insertion unit 20 comprises an outer pipe 30 (also referred to as a sheath pipe) formed substantially in the shape of a pipe parallel to the insertion axis Ax, a protection sheath 32, and an inner sheath 34. The outer pipe 30 forms an outer peripheral wall of the insertion unit 20. An opening of a distal end portion of the outer pipe 30 is inclined from a posture perpendicular to the insertion axis Ax. Further, as described in detail later, a proximal end portion of the outer pipe 30 is connected to the operation part 22 (see FIG. 3).

The protection sheath 32 is inserted into and disposed in the outer pipe 30. A distal end optical system 40 of the camera unit 24 to be described later is provided at a distal end portion of the protection sheath 32. Further, as described in detail later, a proximal end portion of the protection sheath 32 is connected to a case 74 (see FIG. 3) provided in the operation part 22. Furthermore, an insertion passage 31 for the light guide 28 is formed between an inner peripheral surface of the outer pipe 30 and an outer peripheral surface of the protection sheath 32.

The inner sheath 34 corresponds to a shaft member of the present invention, and is inserted into and disposed in the protection sheath 32. The first signal line 26 is inserted into the inner sheath 34. A proximal end optical system 50 and an image pickup unit 60 of the camera unit 24 to be described later are provided at a distal end portion of the inner sheath 34. Further, as described in detail later, a proximal end portion of the inner sheath 34 is connected to a first connection member 90 (see FIG. 3) provided in the operation part 22.

The camera unit 24 comprises the distal end optical system 40, the proximal end optical system 50, and the image pickup unit 60. Reference character OA shown in FIG. 2 denotes the optical axis of the optical system of the camera unit 24.

The distal end optical system 40 is provided at the distal end portion of the protection sheath 32. The distal end optical system 40 is an oblique-viewing optical system that refracts light, which is incident in a direction inclined with respect to the insertion axis Ax, in a direction parallel to the insertion axis Ax and that guides the light to the proximal end optical system 50. The distal end optical system 40 includes a distal end portion body 42 and a distal end lens barrel 44 that is provided in the distal end portion body 42.

The distal end portion body 42 forms a distal end portion of the insertion unit 20 (protection sheath 32) and is a cap (cover) that covers the distal end lens barrel 44. The distal end portion body 42 is formed substantially in the shape of a pipe parallel to the insertion axis Ax. Further, a cover glass 46, which is in an inclined posture corresponding to an inclination angle of an objective lens 48a provided in the distal end lens barrel 44 to be described later, is provided at a distal end-side opening portion of the distal end portion body 42.

Furthermore, the distal end portion body 42 is fixed to the inner peripheral surface of the outer pipe 30. Accordingly, the outer pipe 30, the distal end optical system 40, and the protection sheath 32 are integrally rotated in a direction around the insertion axis Ax (hereinafter, simply abbreviated as a direction around the axis).

The distal end lens barrel 44 houses the objective lens 48a, a prism 48b, and a lens 48c. The objective lens 48a is inclined from a posture perpendicular to the insertion axis Ax and faces the cover glass 46. The objective lens 48a emits light, which is incident through the cover glass 46, toward the prism 48b. The prism 48b corresponds to a second refractive optical element of the present invention, and refracts light incident from the objective lens 48a, that is, light incident in a direction inclined with respect to the insertion axis Ax, in a direction parallel to the insertion axis Ax and then emits the light toward the lens 48c. The lens 48c is in a posture perpendicular to the insertion axis Ax, and emits light incident from the prism 48b toward lenses 56 that are provided in a proximal end lens barrel 52 of the proximal end optical system 50 to be described later.

The configuration of an optical system provided in the distal end lens barrel 44 is not particularly limited as long as light incident in a direction inclined with respect to the insertion axis Ax can be guided into the proximal end lens barrel 52.

A tubular portion 45, which extends toward a proximal end side of the distal end lens barrel 44, is formed at the distal end lens barrel 44. The tubular portion 45 is externally fitted to a distal end portion of the proximal end lens barrel 52 to be described later to be relatively rotatable in the direction around the axis. Accordingly, the proximal end lens barrel 52 is fitted to the distal end lens barrel 44 to be relatively rotatable in the direction around the axis. The tubular portion 45 is formed integrally with the distal end lens barrel 44 in this embodiment, but may be formed separately from the distal end lens barrel 44.

The proximal end optical system 50 is provided at the distal end portion of the inner sheath 34, and guides light, which is incident from the distal end lens barrel 44, to the image pickup unit 60. The proximal end optical system 50 includes the proximal end lens barrel 52, a holder 54, and a prism 55.

The proximal end lens barrel 52 is connected (fixed) to the distal end portion of the inner sheath 34 via the holder 54. A proximal end portion of the proximal end lens barrel 52 may be directly connected to the distal end portion of the inner sheath 34, and the holder 54 may be connected to the proximal end portion of the proximal end lens barrel 52 in the inner sheath 34.

Further, the distal end portion of the proximal end lens barrel 52 is fitted to a proximal end-side opening portion of the tubular portion 45 to be relatively rotatable in the direction around the axis as already described. Accordingly, one of the distal end lens barrel 44 and the proximal end lens barrel 52 is rotatable relative to the other thereof in the direction around the axis. A proximal end portion of the distal end lens barrel 44 may be fitted into a distal end-side opening portion of the proximal end lens barrel 52 to be relatively rotatable in the direction around the axis.

A plurality of lenses 56 having an optical axis OA parallel to the insertion axis Ax are provided in the proximal end lens barrel 52. Each lens 56 emits light, which is incident from the distal end lens barrel 44, toward the prism 55.

The holder 54 is formed substantially in the shape of a pipe parallel to the insertion axis Ax, and is fixed to the distal end portion of the inner sheath 34. Further, the holder 54 is connected and fixed (externally fitted and fixed) to the proximal end portion of the proximal end lens barrel 52. Accordingly, since the inner sheath 34 and the proximal end lens barrel 52 are connected to each other by the holder 54, the inner sheath 34, the proximal end lens barrel 52, and the holder 54 are integrally rotated in the direction around the axis.

The prism 55 is held at a proximal end-side opening portion of the holder 54, and the image pickup unit 60 to be described later is held via the prism 55. For this reason, the image pickup unit 60 is rotated in the direction around the axis integrally with the inner sheath 34 and the proximal end lens barrel 52 via the holder 54 and the prism 55.

The prism 55 corresponds to a first refractive optical element of the present invention, and is held at the proximal end-side opening portion of the holder 54 as already described. The prism 55 refracts light, which is incident through the proximal end lens barrel 52, by an angle of 90°. A mirror may be used instead of the prism 55.

The image pickup unit 60 picks up the image of light that passes through the distal end lens barrel 44 and the proximal end lens barrel 52 and that is reflected by the prism 55. The image pickup unit 60 comprises an image pickup element 64 and a circuit board 66.

The image pickup element 64 is connected (fixed) to the prism 55 in a state where the image pickup element 64 is mounted on the circuit board 66, and is mounted on the holder 54 via the prism 55. Accordingly, the holder 54 corresponds to an image pickup element-mounting part of the present invention. Further, the image pickup element 64 picks up the image of the light, which is refracted by the prism 55, and outputs image pickup signals. A charge coupled device (CCD) image sensor or a complementary metal oxide semiconductor (CMOS) image sensor is used as the image pickup element 64.

The image pickup element 64 is mounted on the holder 54 via the prism 55 in this embodiment, but the image pickup element 64 may be directly mounted on the proximal end-side opening portion of the holder 54. In this case, the image pickup element 64 is held in a posture perpendicular to the insertion axis Ax (optical axis OA) by the holder 54 and has a light-receiving surface orthogonal to the optical axis OA.

The circuit board 66 controls the drive of the image pickup element 64. Further, the distal end portion of the first signal line 26 is connected to the circuit board 66 via a connector 68. Furthermore, the circuit board 66 outputs the image pickup signals of the image pickup element 64 to the first signal line 26 via the connector 68.

FIG. 3 is a cross-sectional view of the operation part 22. As shown in FIG. 3, the operation part 22 is an operation ring formed substantially in the shape of a pipe parallel to the insertion axis Ax, and receives a rotating operation that is performed in the direction around the axis by a practitioner.

A proximal end portion of the already-described outer pipe 30 is connected to a distal end portion of the operation part 22. Accordingly, in a case where the operation part 22 is operated to be rotated in the direction around the axis, the protection sheath 32 and the distal end optical system 40 (the distal end portion body 42 and the distal end lens barrel 44) are rotated in the same direction via the outer pipe 30. Therefore, the visual field direction (observation direction) of the oblique-viewing endoscope 10 can be rotated.

The proximal end portions of the protection sheath 32 and the inner sheath 34 are inserted into a distal end-side opening portion of the operation part 22. Further, an extending part 72 is provided in a proximal end-side opening portion of the operation part 22. Furthermore, the case 74 is provided in the operation part 22.

The extending part 72 is formed substantially in the shape of a pipe parallel to the insertion axis Ax to have a diameter smaller than an inner diameter of the operation part 22. An O-ring 76 is externally fitted to an outer peripheral surface of a distal end portion of the extending part 72. Further, the distal end portion of the extending part 72 is rotatably held on an inner peripheral surface of a proximal end portion of the operation part 22 via the O-ring 76. Accordingly, the extending part 72 is held by the proximal end portion of the operation part 22 to be relatively rotatable in the direction around the axis. As a result, in a case where a rotational force for rotating the operation part 22 in the direction around the axis is applied to the operation part 22, this rotational force is not transmitted to the extending part 72.

Further, an extension part 78 is inserted into the extending part 72. The extension part 78 is formed substantially in the shape of a pipe parallel to the insertion axis Ax, and the second signal line 27 is inserted into the extension part 78. A proximal end portion of the extension part 78 is fixed to the extending part 72 via a fixing member 79. Accordingly, the extending part 72 and the extension part 78 are integrated with each other. Further, as described in detail later, a distal end portion of the extension part 78 is connected to a magnet coupling 102 via a second connection member 100 and a second bearing receiving member 96.

The case 74 is formed substantially in the shape of a pipe parallel to the insertion axis Ax to have a diameter smaller than the inner diameter of the operation part 22 and is housed in the operation part 22. The case 74 is supported in the internal space of the operation part 22 by the protection sheath 32, the extension part 78, and the like. A distal end side of the case 74 is connected to the proximal end portion of the protection sheath 32. Accordingly, the case 74 is rotated integrally with the protection sheath 32 in the direction around the axis. As a result, in a case where a rotational force for rotating the operation part 22 in the direction around the axis is applied to the operation part 22, this rotational force is transmitted to the outer pipe 30, the distal end optical system 40, the protection sheath 32, and the case 74, so that these components are rotated in the same direction as the operation part 22.

The proximal end portion of the inner sheath 34 and the proximal end portion of the first signal line 26 are disposed in the case 74. Further, a partition wall 74a perpendicular to the insertion axis Ax is provided in the case 74, for example, in a proximal end-side opening portion of the case 74. This partition wall 74a closes the proximal end-side opening portion of the case 74.

A tubular portion 74b parallel to the insertion axis Ax is provided on a proximal end side of the case 74. The tubular portion 74b is formed to have the same diameter as the case 74 in this embodiment, but may be formed to have a diameter different from the diameter of the case 74. Further, the tubular portion 74b may be formed integrally with the case 74. In this case, a proximal end portion of the case 74 functions as the tubular portion 74b. The distal end portion of the second signal line 27 is disposed in the tubular portion 74b in addition to a part of a connecting unit 84 to be described later.

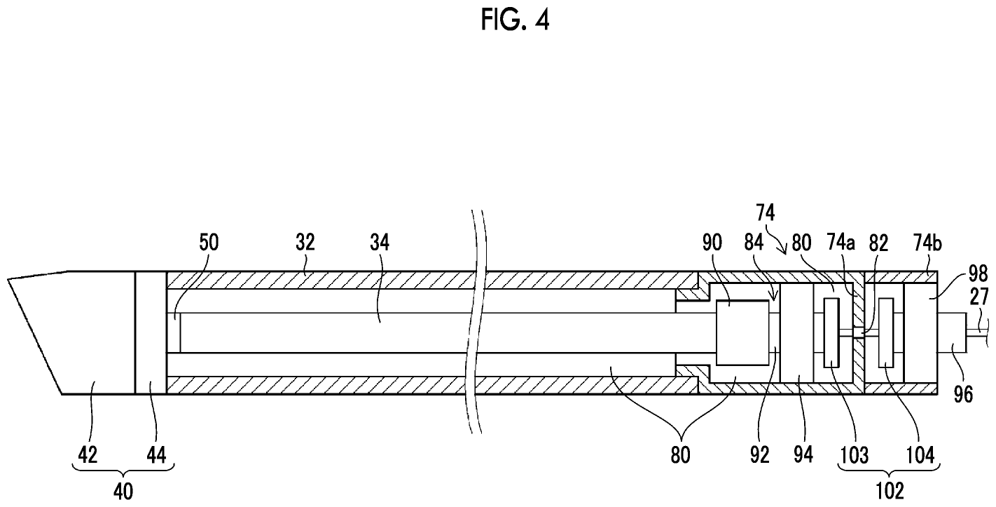

FIG. 4 is a cross-sectional view of the protection sheath 32 and the case 74. A sealed space 80 (airtight space) is formed in the protection sheath 32 and the case 74 as shown in FIG. 4, and the inner sheath 34, the image pickup unit 60, the first signal line 26, and the like are disposed in the sealed space 80. A distal end side of the sealed space 80 is defined by the distal end optical system 40. Further, a proximal end side of the sealed space 80 is defined by the partition wall 74a. Accordingly, the moisture-proof property of the camera unit 24 is improved, so that fogging is prevented.

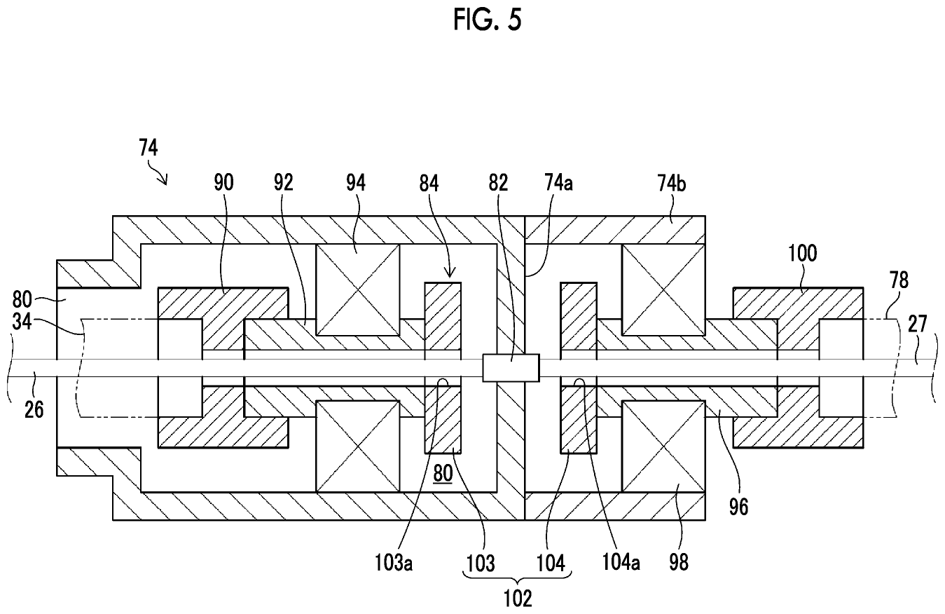

FIG. 5 is an enlarged cross-sectional view of the case 74 and the tubular portion 74b. As shown in FIGS. 3 to 5, the already-described partition wall 74a, an airtight connector 82, and a connecting unit 84 are provided in the case 74 and the tubular portion 74b.

The airtight connector 82 is provided to pass through the inside and outside of the sealed space 80 and to be rotatable relative to the partition wall 74a in the direction around the axis. The airtight connector 82 electrically connects the first signal line 26 provided in the case 74 (in the sealed space 80) to the second signal line 27 provided in the tubular portion 74b (outside the sealed space 80). In a case where the first signal line 26 and the second signal line 27 are torsionally deformable in the direction around the axis, the airtight connector 82 may be fixed to the partition wall 74a.

The connecting unit 84 is provided in the case 74 and the tubular portion 74b to be rotatable relative to the case 74 and the tubular portion 74b in the direction around the axis. The first signal line 26 and the second signal line 27 are inserted into the connecting unit 84. The connecting unit 84 magnetically connects the proximal end portion of the inner sheath 34 provided in the case 74 (in the sealed space 80) to the distal end portion of the extension part 78 provided outside the sealed space 80 with the partition wall 74a interposed therebetween.

The connecting unit 84 comprises a first connection member 90, a first bearing receiving member 92, a first bearing 94, a second bearing receiving member 96, a second bearing 98, a second connection member 100, and a magnet coupling 102.

The first connection member 90 and the first bearing receiving member 92 are provided in the case 74 (in the sealed space 80), and are formed substantially in the shape of a pipe parallel to the insertion axis Ax. Further, the first signal line 26 is inserted into the first connection member 90 and the first bearing receiving member 92.

The first connection member 90 connects the proximal end portion of the inner sheath 34 to the first bearing receiving member 92 in the case 74 (in the sealed space 80). Accordingly, the first bearing receiving member 92 is connected to a proximal end side of the inner sheath 34 via the first connection member 90.

A distal end side of the first bearing receiving member 92 is connected to the first connection member 90 as described above, and a proximal end side thereof is fixed to a first magnet 103 of the magnet coupling 102 to be described later. Further, the first bearing 94, which is inscribed in the case 74, is fixed to an outer peripheral surface of the first bearing receiving member 92. Accordingly, the first bearing receiving member 92 and the first magnet 103 are held in the case 74 to be rotatable relative to the case 74 in the direction around the axis. Publicly known various radial bearings, such as a ball bearing and a roller bearing, are used as the first bearing 94.

The second bearing receiving member 96 is provided in the tubular portion 74b (outside the sealed space 80), and the second connection member 100 is provided between the second bearing receiving member 96 and the extension part 78. The second bearing receiving member 96 and the second connection member 100 are formed substantially in the shape of a pipe parallel to the insertion axis Ax, and the second signal line 27 is inserted into each of the second bearing receiving member 96 and the second connection member 100.

A distal end portion of the second bearing receiving member 96 is fixed to a second magnet 104 of the magnet coupling 102 (to be described later) in the tubular portion 74b, and a proximal end portion thereof is connected to the second connection member 100. Further, the second bearing 98, which is inscribed in the tubular portion 74b, is fixed to an outer peripheral surface of the second bearing receiving member 96. Accordingly, the second bearing receiving member 96 and the second magnet 104 are held in the tubular portion 74b to be rotatable relative to the tubular portion 74b in the direction around the axis. Publicly known various radial bearings are also used as the second bearing 98 as in the case of the first bearing 94.

The second connection member 100 connects the second bearing receiving member 96 to the distal end portion of the extension part 78. Accordingly, the second bearing receiving member 96 is connected to the distal end side of the extension part 78 via the second connection member 100.

The magnet coupling 102 includes the first magnet 103 provided in the case 74 (in the sealed space 80) and the second magnet 104 provided in the tubular portion 74b (outside the sealed space 80) with the partition wall 74a interposed therebetween. The magnet coupling 102 is a magnetic connecting member that magnetically connects the first bearing receiving member 92 (inner sheath 34) to the second bearing receiving member 96 (extension part 78).

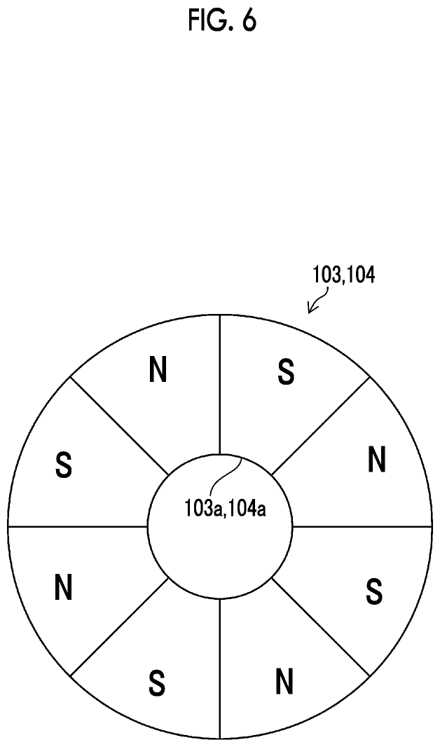

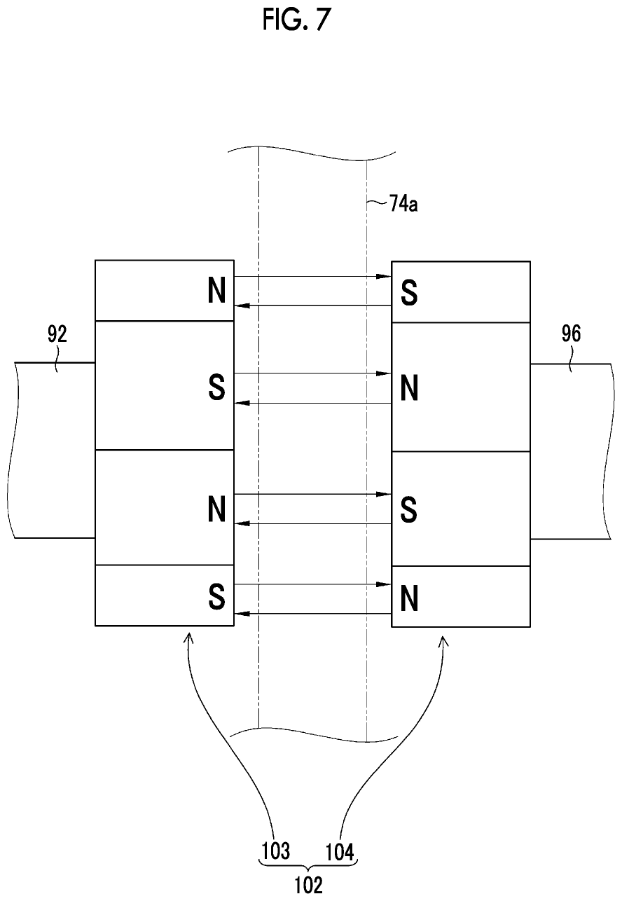

FIG. 6 is a front view of the first magnet 103 and the second magnet 104 as viewed from a partition wall 74a side. FIG. 7 is a side view of the first magnet 103 and the second magnet 104. As shown in FIG. 6, the first magnet 103 and the second magnet 104 have the shape of a disk (the shape of a ring) parallel to the partition wall 74a (perpendicular to the insertion axis Ax). An insertion hole 103a into which the first signal line 26 is to be inserted is formed at a central portion of the first magnet 103, and an insertion hole 104a into which the second signal line 27 is to be inserted is formed at a central portion of the second magnet 104. Further, each of the first magnet 103 and the second magnet 104 of this embodiment is a so-called single-sided multi-pole magnet, and a plurality of sets of N poles and S poles are formed on the surface side thereof facing the partition wall 74a at regular angular intervals in the direction around the axis.

Each of the first magnet 103 and the second magnet 104 is not limited to a single-sided multi-pole magnet and may be a double-sided multi-pole magnet, and the number of poles is not particularly limited as long as the number is two or more. Furthermore, the shape of each of the first magnet 103 and the second magnet 104 is not limited to the shape of a disk, and the shape of any member parallel to the partition wall 74a, such as a polygonal shape, may be employed.

As shown in FIG. 7, the first magnet 103 and the second magnet 104 are disposed with the partition wall 74a interposed therebetween such that individual N poles of any one of the first magnet 103 or the second magnet 104 face individual S poles of the other thereof and individual S poles of one thereof face individual N poles of the other thereof. Accordingly, the first magnet 103 and the second magnet 104 are magnetically connected to each other in a thrust direction of the insertion axis Ax [a direction parallel to the insertion axis Ax (axial direction)] with the partition wall 74a interposed therebetween. As a result, the inner sheath 34 and the extending part 72 are magnetically connected to each other via the magnet coupling 102.

Since the inner sheath 34 and the extending part 72 are magnetically connected to each other via the magnet coupling 102, torque (stop torque, rotational torque) can be transmitted to the inner sheath 34 from the extending part 72. Accordingly, in a case where a practitioner rotationally operates the operation part 22, the rotation of the inner sheath 34 (the proximal end optical system 50 and the image pickup unit 60) and of the protection sheath 32 in the direction around the axis is prevented, that is, the posture of the inner sheath 34 in the direction around the axis is maintained by the magnet coupling 102. On the other hand, in a case where a practitioner rotationally operates the extending part 72, it is possible to rotate the inner sheath 34 in the direction around the axis by means of the magnet coupling 102. In addition, in a case where a practitioner rotates the operation part 22 or the extending part 72, the occurrence of blurring in an observation image caused by the eccentricity of the image pickup unit 60 and the like from the insertion axis Ax is prevented.

Since the magnet coupling 102 acting in the thrust direction is used in the oblique-viewing endoscope 10 according to the first embodiment as described above, an increase in diameter of the operation part 22 is prevented as compared to a case where the concentric circular magnet coupling (the related art) disclosed in US2019/0117048A is used.

Further, since the magnet coupling 102 of the first embodiment can include two magnets, that is, the first magnet 103 and the second magnet 104, the number of magnets can be reduced as compared to the related art in which a lot of magnets need to be arranged concentrically. As a result, it is possible to prevent complicated assembly work of the operation part 22 and to reduce costs.

### Second Embodiment

In the magnet coupling 102 of the first embodiment, a plurality of N poles and S poles are alternately formed in each of the first magnet 103 and the second magnet 104 in the direction around the axis as shown in the already-described FIG. 6. For this reason, a magnetic force is weakened at a boundary portion between the N pole and the S pole. As a result, in the magnet coupling 102 of the first embodiment, there is a concern that transmission torque, which is torque (stop torque, rotational torque) to be transmitted from one of the first magnet 103 and the second magnet 104 to the other thereof, may be weakened.

In this case, the number of poles of each of the first magnet 103 and the second magnet 104 can be reduced to increase transmission torque, but the amount of slip between the first magnet 103 and the second magnet 104 that occurs in a case where transmission torque equal to or larger than allowable torque is transmitted from one of the first magnet 103 and the second magnet 104 to the other thereof is increased. On the other hand, in a case where each of the first magnet 103 and the second magnet 104 is multipolar, the above-mentioned amount of slip is reduced, but transmission torque is reduced.

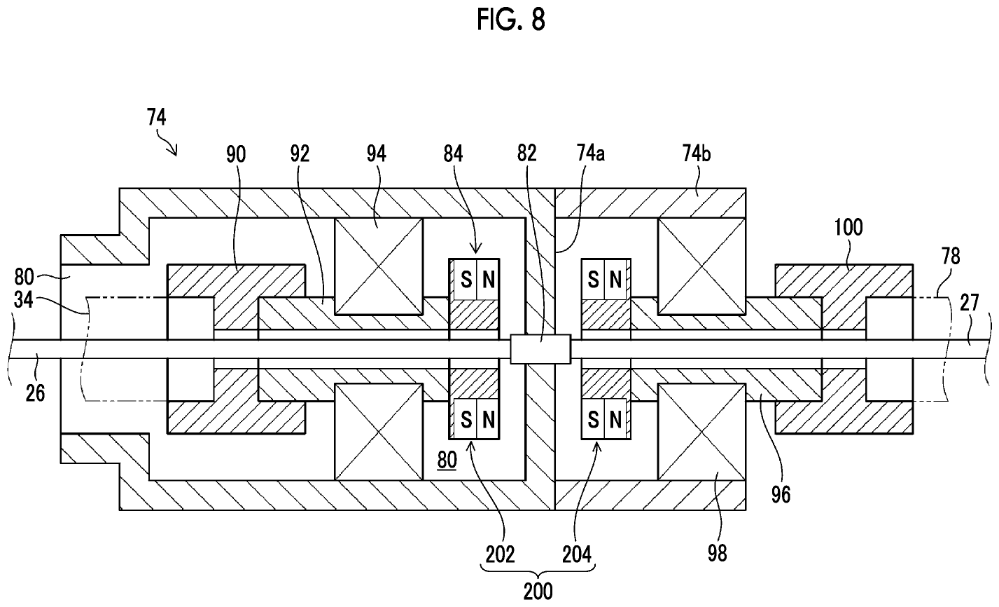

Accordingly, an oblique-viewing endoscope 10 according to a second embodiment comprises a magnet coupling 200 (see FIG. 8), which is different from the magnet coupling 102 of the first embodiment, in order to achieve both an improvement in transmission torque and a reduction in the amount of slip.

FIG. 8 is an enlarged cross-sectional view of a case 74 and a tubular portion 74b that are provided in an operation part 22 of the oblique-viewing endoscope 10 according to the second embodiment (corresponding to the endoscope of the present invention). The oblique-viewing endoscope 10 according to the second embodiment has basically the same configuration as the oblique-viewing endoscope 10 according to the first embodiment except that the magnet coupling 200 is provided instead of the magnet coupling 102. For this reason, components having the same functions or configuration as in the first embodiment will be denoted by the same reference numerals as those of the first embodiment, and the description thereof will be omitted.

The magnet coupling 200 includes a first magnet 202 provided in the case 74 (in the sealed space 80) and a second magnet 204 provided in the tubular portion 74b (outside the sealed space 80) with the partition wall 74a interposed therebetween. The magnet coupling 200 magnetically connects the first bearing receiving member 92 (inner sheath 34) to the second bearing receiving member 96 (extension part 78) as in the first embodiment.

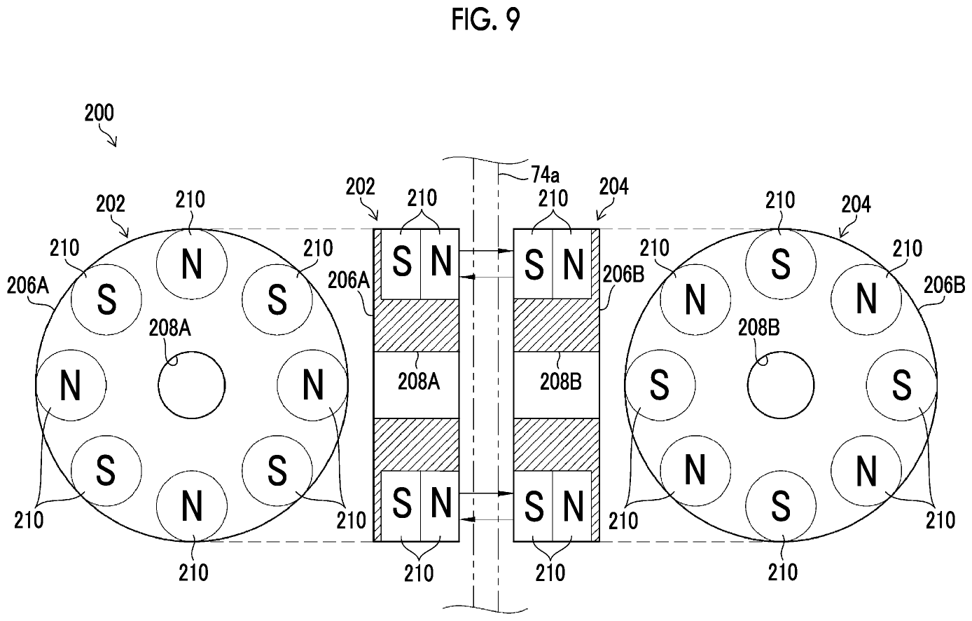

FIG. 9 shows enlarged cross-sectional views of the first magnet 202 and the second magnet 204 shown in FIG. 8, and enlarged front views of the first magnet 202 and the second magnet 204 as viewed from a partition wall 74a side, respectively.

As shown in FIG. 9 and in the already-described FIG. 8, the first magnet 202 comprises a magnet holding part 206A and eight individual magnets 210. The second magnet 204 has basically the same configuration as the first magnet 202, and comprises a magnet holding part 206B and eight individual magnets 210.

The magnet holding parts 206A and 206B are formed in an annular shape as viewed in an axial direction of the insertion axis Ax, that is, as viewed from the partition wall 74a side. For example, a non-magnetic material is used as material of these magnet holding parts 206A and 206B. The magnet holding part 206A is fixed to a proximal end side of the first bearing receiving member 92 (inner sheath 34), and includes an insertion hole 208A into which the first signal line 26 is to be inserted and which is formed at a central portion thereof. Further, the magnet holding part 206B is fixed to a distal end side of the second bearing receiving member 96, and includes an insertion hole 208B into which the second signal line 27 is to be inserted and which is formed at a central portion thereof.

Furthermore, fitting holes (not shown) to which eight individual magnets 210 are to be fitted, respectively, are formed on a facing surface (hereinafter, referred to as a partition wall-facing surface) of each of the magnet holding parts 206A and 206B facing the partition wall 74a at regular intervals in the direction around the insertion axis Ax.

The individual magnet 210 is a needle-like magnet or a rod-like magnet that extends in a direction parallel to the insertion axis Ax. For example, in the second embodiment, the individual magnet 210 is formed in a columnar shape (including a substantially columnar shape), and is formed such that the length (total length) of the individual magnet in the axial direction of the insertion axis Ax is larger than the diameter of the individual magnet. Further, since the individual magnet 210 has magnetic poles in the axial direction of the insertion axis Ax, that is, is divided into an N pole and an S pole in the axial direction of the insertion axis Ax, magnetic forces on both end surfaces of the individual magnet in the axial direction of the insertion axis Ax are largest.

Eight individual magnets 210 are provided on each of the magnet holding parts 206A and 206B at regular intervals in the direction around the axis of each magnet holding part. In this case, the respective individual magnets 210 are provided in each of the magnet holding parts 206A and 206B such that the magnetic poles of one of the individual magnets 210 adjacent to each other in the direction around the axis are reversed with respect to the magnetic poles of the other thereof, that is, the magnetic poles of the individual magnets 210 are alternately reversed in the direction around the axis. Further, end surfaces of the respective individual magnets 210 facing the partition wall 74a are exposed on the partition wall-facing surfaces of the magnet holding parts 206A and 206B.

End surfaces of N poles of four individual magnets 210 of the magnet holding part 206A and end surfaces of S poles of four individual magnets 210 of the magnet holding part 206B are disposed to face each other with the partition wall 74a interposed therebetween. Further, at the same time, end surfaces of S poles of four individual magnets 210 of the magnet holding part 206A and end surfaces of N poles of four individual magnets 210 of the magnet holding part 206B are disposed to face each other with the partition wall 74a interposed therebetween. Accordingly, since the first magnet 202 and the second magnet 204 are magnetically connected to each other with the partition wall 74a interposed therebetween as in the first embodiment, the inner sheath 34 and the extending part 72 are magnetically connected to each other via the magnet coupling 200.

Since the plurality of individual magnets 210 are provided on the magnet holding parts 206A and 206B at intervals in the direction around the axis in the magnet coupling 200 of the second embodiment as described above, the weakening of a magnetic force between the N poles and the S poles exposed on the partition wall-facing surfaces is reduced as compared to the first embodiment. Further, end surfaces of the respective individual magnets 210 where a magnetic force is strongest can be exposed on the partition wall-facing surfaces of the magnet holding parts 206A and 206B in the magnet coupling 200 of the second embodiment. Accordingly, transmission torque can be improved in the magnet coupling 200 of the second embodiment as compared to a case where N poles and S poles are alternately formed in one magnet (the first magnet 103 and the second magnet 104) in the direction around the axis as in the first embodiment.

At this time, it is preferable that the respective individual magnets 210 are provided eccentrically to the outer peripheral side from the inner peripheral side of the magnet holding parts 206A and 206B in a case where the magnet holding parts 206A and 206B are viewed from the partition wall 74a side. Accordingly, transmission torque to be transmitted from one of the first magnet 202 and the second magnet 204 to the other thereof can be further improved.

Further, transmission torque is improved in the magnet coupling 200 of the second embodiment. Accordingly, in a case where a practitioner rotationally operates the operation part 22, the rotation of the inner sheath 34 (the proximal end optical system 50 and the image pickup unit 60) and of the protection sheath 32 in the direction around the axis is reliably prevented. On the other hand, in a case where a practitioner rotationally operates the extending part 72, the inner sheath 34 (the proximal end optical system 50 and the image pickup unit 60) can be rotated in the direction around the axis without delay.

Furthermore, in the magnet coupling 200 of the second embodiment, transmission torque can be improved without a reduction in the number of poles as in the magnet coupling 102 of the first embodiment. Accordingly, the amount of slip occurring in a case where transmission torque equal to or larger than allowable torque is transmitted from one of the first magnet 202 and the second magnet 204 to the other thereof can also be reduced. As a result, the oblique-viewing endoscope 10 according to the second embodiment can achieve both an improvement in transmission torque and a reduction in the amount of slip by the magnet coupling 200.

### Modification Examples 1 and 2 of First Magnet and Second Magnet of Second Embodiment

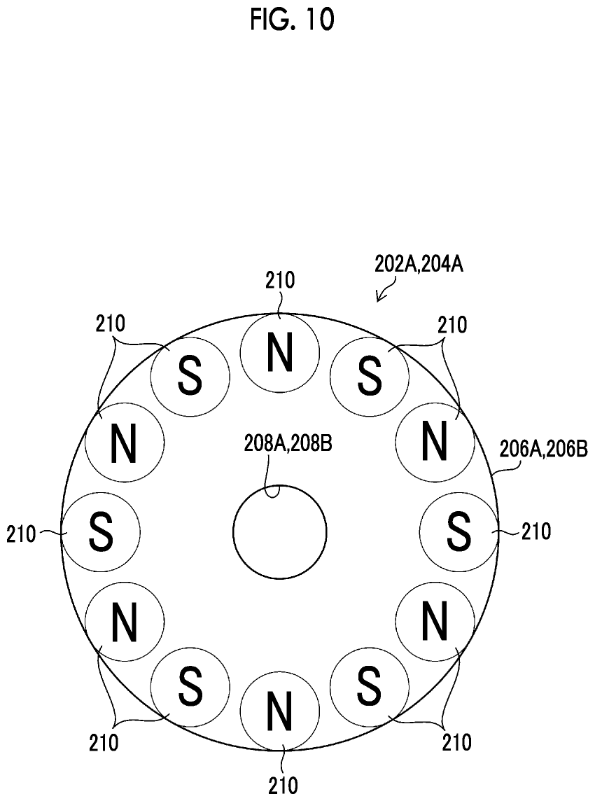

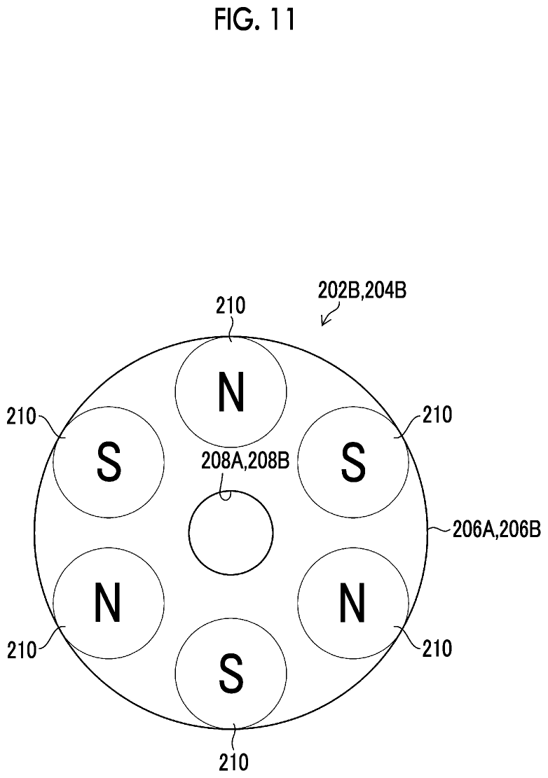

FIG. 10 is a front view of a first magnet 202A and a second magnet 204A of Modification Example 1 of the second embodiment as viewed from a partition wall 74a side, respectively. FIG. 11 is a front view of a first magnet 202B and a second magnet 204B of Modification Example 2 of the second embodiment as viewed from a partition wall 74a side, respectively.

Each of the first magnet 202 and the second magnet 204 comprises eight individual magnets 210 in the second embodiment, but the number of individual magnets 210 is not particularly limited. For example, the number of individual magnets 210 provided on each of the first magnet 202A and the second magnet 204A may be increased from eight as shown in FIG. 10, or the number of individual magnets 210 provided on each of the first magnet 202B and the second magnet 204B may be reduced from eight as shown in FIG. 11.

### Modification Examples 3 and 4 of First Magnet and Second Magnet of Second Embodiment

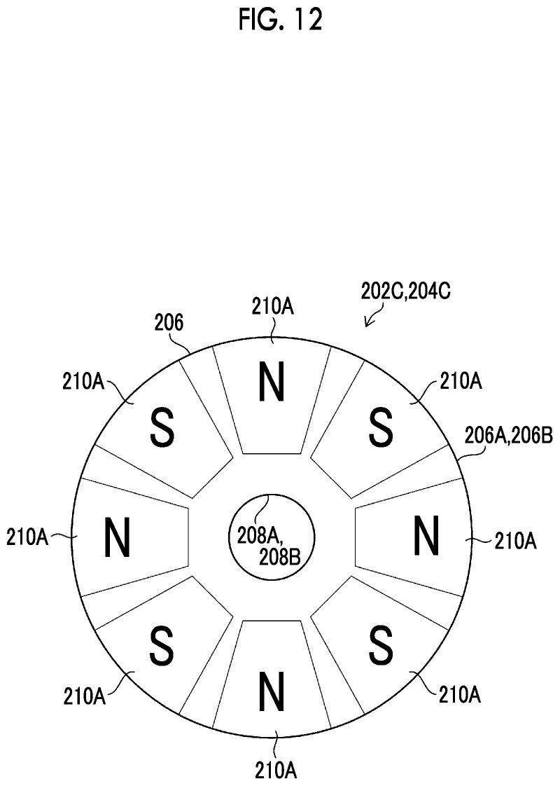

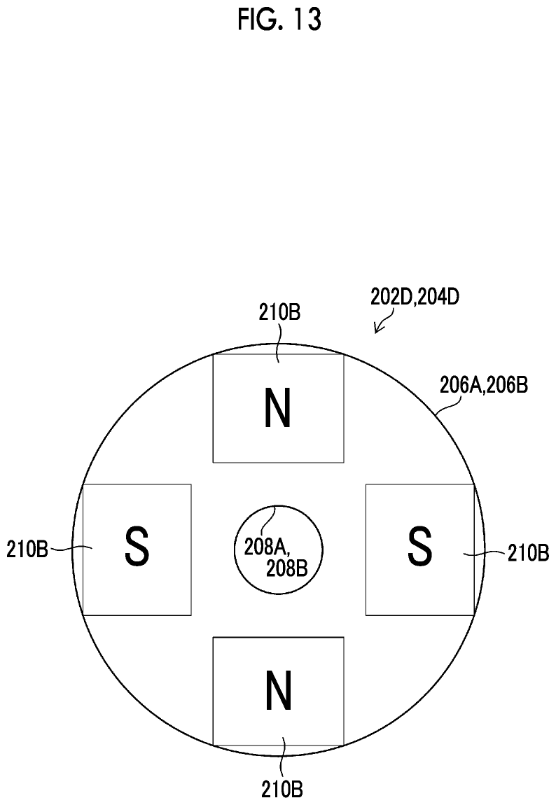

FIG. 12 is a front view of a first magnet 202C and a second magnet 204C of Modification Example 3 of the second embodiment as viewed from a partition wall 74a side, respectively. FIG. 13 is a front view of a first magnet 202D and a second magnet 204D of Modification Example 4 of the second embodiment as viewed from a partition wall 74a side, respectively.

Each of the first magnet 202 and the second magnet 204 comprises a plurality of individual magnets 210 having a columnar shape in the second embodiment, but the shape of each individual magnet 210 is not particularly limited. For example, a plurality of individual magnets 210A having a substantially trapezoidal columnar shape may be provided on each of the first magnet 202C and the second magnet 204C as shown in FIG. 12, or a plurality of individual magnets 210B having a substantially square columnar shape may be provided on each of the first magnet 202D and the second magnet 204D as shown in FIG. 13.

### Modification Example 5 of First Magnet and Second Magnet of Second Embodiment

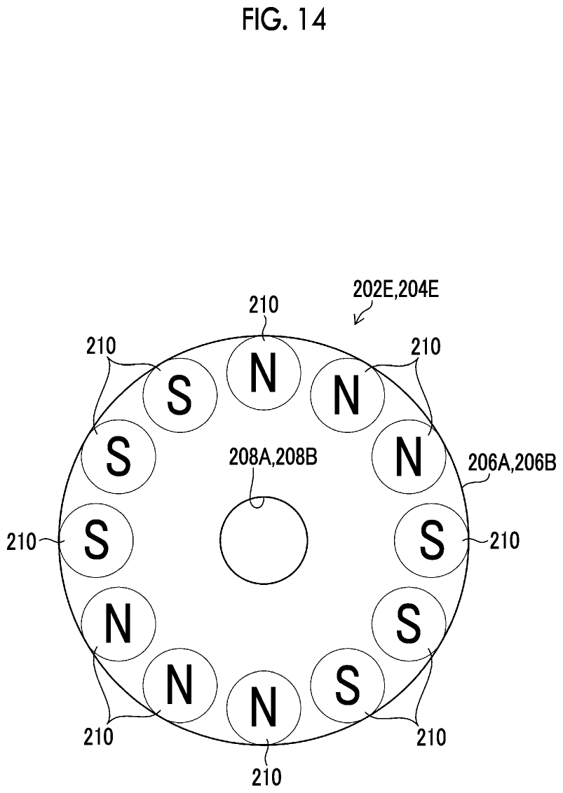

FIG. 14 is a front view of a first magnet 202E and a second magnet 204E of Modification Example 5 of the second embodiment as viewed from a partition wall 74a side, respectively.

The magnetic poles of the respective individual magnets 210 provided on each of the magnet holding parts 206A and 206B are alternately reversed in the direction around the axis in the second embodiment, but the present invention is not limited thereto. For example, the magnetic poles of some individual magnets 210 adjacent to each other in the direction around the axis on the partition wall-facing surface may be the same as shown in FIG. 14. In Modification Example 5, the respective individual magnets 210 are provided on each of the magnet holding parts 206A and 206B such that a plurality of (here, three) individual magnets 210 are regarded as one magnet group and the magnetic poles of the magnet groups are alternately reversed in the direction around the axis.

### Other Modification Examples of Second Embodiment

The respective individual magnets 210 are provided eccentrically to the outer peripheral side of the magnet holding parts 206A and 206B in the second embodiment, but may be provided eccentrically to the inner peripheral side of the magnet holding parts 206A and 206B or may be provided at middle positions between an inner periphery and an outer periphery of the magnet holding parts 206A and 206B.

In the second embodiment, the magnet holding parts 206A and 206B are formed in an annular shape, and a plurality of individual magnets 210 are provided on the magnet holding parts 206A and 206B at regular intervals in the direction around the axis. However, the shapes of the magnet holding parts 206A and 206B and the arrangement pattern of the individual magnets 210 are not particularly limited and can be changed as appropriate.

[Other]

The hollow inner sheath 34 has been described in each embodiment as the shaft member of the present invention by way of example, but a solid shaft member may be inserted into the protection sheath 32.

In each embodiment, the inner sheath 34 and the first bearing receiving member 92 are connected to each other via the first connection member 90, and the extension part 78 and the second bearing receiving member 96 are connected to each other via the second connection member 100. However, the first bearing receiving member 92 may be directly connected to the inner sheath 34, and the second bearing receiving member 96 may be directly connected to the extension part 78.

In each embodiment, the extending part 72 is provided at the proximal end portion of the operation part 22 to be relatively rotatable, and the extension part 78 is inserted into the extending part 72. However, the extending part 72 and the extension part 78 may be integrally molded.

The case 74 is provided with the tubular portion 74b in each embodiment, but the tubular portion 74b may be omitted. In this case, the second bearing receiving member 96 and the second bearing 98 are omitted, and the distal end portion of the extension part 78 is connected to the second magnet 104.

A rigid endoscope has been described in each embodiment as the oblique-viewing endoscope 10 by way of example, but the present invention can be applied even in the case of a flexible endoscope. Further, the oblique-viewing endoscope 10 has been described in the embodiments as the endoscope of the present invention by way of example, but the present invention can be applied to various endoscopes in which a protection sheath can be rotated relative to an inner sheath in a direction around an axis according to an operation for rotating an operation part.

### EXPLANATION OF REFERENCES

10: oblique-viewing endoscope
12: endoscope system
14: processor device
16: monitor
18: light source device
20: insertion unit
22: operation part
24: camera unit
26: first signal line
27: second signal line
28: light guide
30: outer pipe
31: insertion passage
32: protection sheath
34: inner sheath
40: distal end optical system
42: distal end portion body
44: distal end lens barrel
45: tubular portion
46: cover glass
48a: objective lens
48b: prism
48c: lens
50: proximal end optical system
52: proximal end lens barrel
54: holder
55: prism
56: lens
60: image pickup unit
64: image pickup element
66: circuit board
68: connector
72: extending part
74: case
74a: partition wall
74b: tubular portion
76: O-ring
78: extension part
79: fixing member
80: sealed space
82: airtight connector
84: connecting unit
90: first connection member
92: first bearing receiving member
94: first bearing
96: second bearing receiving member
98: second bearing
100: second connection member
102: magnet coupling
103: first magnet
103a: insertion hole
104: second magnet
104a: insertion hole
200: magnet coupling
202, 202A to 202E: first magnet
204, 204A to 204E: second magnet
206A, 206B: magnet holding part
208A, 208B: insertion hole
210, 210A, 210B: individual magnet
Ax: insertion axis
OA: optical axis

What is claimed is:

1. An endoscope comprising:
a protection sheath that forms an insertion unit;
a pipe-like case that is connected to a proximal end side of the protection sheath;
a distal end optical system that is provided at a distal end of the protection sheath and that defines a distal end side of a sealed space formed in the protection sheath and the case;
a partition wall that is provided in the case, is perpendicular to an insertion axis of the insertion unit, and defines a proximal end side of the sealed space;
a shaft member that is inserted into the protection sheath and that is rotatable relative to the protection sheath in a direction around the insertion axis;
an image pickup unit that is provided at a distal end of the shaft member and that picks up an image of light passing through the distal end optical system; and
a magnet coupling which includes a first magnet provided in the sealed space and a second magnet provided outside the sealed space with the partition wall interposed therebetween and of which the first magnet is connected to a proximal end side of the shaft member,
wherein the magnet coupling and the case are rotatable relative to each other in the direction around the axis.

2. The endoscope according to claim 1, further comprising:
a signal line that is connected to the image pickup unit,
wherein each of the first magnet and the second magnet is formed in a shape of a disk perpendicular to the insertion axis and includes an insertion hole into which the signal line is to be inserted.

3. The endoscope according to claim 1, further comprising:
a signal line that is connected to the image pickup unit,
wherein each of the first magnet and the second magnet includes a magnet holding part that includes an insertion hole into which the signal line is to be inserted and is formed in an annular shape as viewed from the partition wall, and a plurality of individual magnets that are provided on the magnet holding part at intervals and that have magnetic poles in an axial direction of the insertion axis, and
the magnet holding part of the first magnet is connected to the proximal end side of the shaft member.

4. The endoscope according to claim 3,
wherein the plurality of individual magnets are provided on the magnet holding part in the direction around the axis.

5. The endoscope according to claim 4,
wherein the plurality of individual magnets are provided on the magnet holding part at regular intervals in the direction around the axis.

6. The endoscope according to claim 4,
wherein magnetic poles of one of the individual magnets adjacent to each other in the direction around the axis are reversed with respect to magnetic poles of the other of the individual magnets.

7. The endoscope according to claim 4,
wherein the plurality of individual magnets are eccentric to an outer peripheral side of the magnet holding part in a case where the magnet holding part is viewed from the partition wall.

8. The endoscope according to claim 3,
wherein the individual magnet has a shape extending in a direction parallel to the insertion axis.

9. The endoscope according to claim 2,
wherein the signal line includes a first signal line disposed in the sealed space and a second signal line disposed outside the sealed space, and
the endoscope further comprises an airtight connector that is provided in the partition wall and that connects the first signal line to the second signal line.

10. The endoscope according to claim 2,
wherein the shaft member is an inner sheath into which the signal line is inserted,
the endoscope further comprises a proximal end optical system that is provided at a distal end of the inner sheath and that guides light, which passes through the distal end optical system, to the image pickup unit,
the image pickup unit includes an image pickup element that picks up an image of light incident through the proximal end optical system and that outputs an image pickup signal to the signal line, and
the distal end optical system, the proximal end optical system, and the image pickup element are rotatable relative to each other in the direction around the axis.

11. The endoscope according to claim 10,
wherein the proximal end optical system includes a proximal end lens barrel that is connected to the distal end of the inner sheath, and an image pickup element-mounting part which is connected to a proximal end side of the proximal end lens barrel and on which the image pickup element is mounted.

12. The endoscope according to claim 1,
wherein the distal end optical system includes a distal end portion body and a distal end lens barrel fixed to the distal end portion body.

13. The endoscope according to claim 12,
wherein the shaft member is an inner sheath,
the endoscope further comprises a proximal end optical system that is provided at a distal end of the inner sheath and that guides light, which passes through the distal end optical system, to the image pickup unit,
the proximal end optical system includes a proximal end lens barrel that is connected to the distal end of the inner sheath, and
one of the distal end lens barrel and the proximal end lens barrel is fitted to the other of the distal end lens barrel and the proximal end lens barrel to be relatively rotatable in the direction around the axis.

14. The endoscope according to claim 1, further comprising:
a first bearing receiving member that is fixed to the first magnet in the sealed space of the case; and
a first bearing that is fixed to the first bearing receiving member and that is inscribed in the case,
wherein the magnet coupling and the case are rotatable relative to each other in the direction around the axis via the first bearing.

15. The endoscope according to claim 14,
wherein the proximal end side of the shaft member is connected to the first bearing receiving member.

16. The endoscope according to claim 1, further comprising:
an outer pipe into which the protection sheath is inserted; and
a light guide that is disposed in a space between the outer pipe and the protection sheath.

17. The endoscope according to claim 16,
wherein the distal end optical system is fixed to a distal end side of the outer pipe,
the endoscope further comprises an operation part that is connected to a proximal end side of the outer pipe and that houses the case, and
in a case where a rotational force for rotating the operation part in the direction around the axis is applied to the operation part, the rotational force is transmitted to the protection sheath and to the case via the outer pipe and the distal end optical system.

18. The endoscope according to claim 17, further comprising:
a tubular portion that is provided on a proximal end side of the case;
a second bearing receiving member that is fixed to the second magnet outside the sealed space;
a second bearing that is fixed to the second bearing receiving member and that is inscribed in the tubular portion;
a pipe-like extending part that is provided on a proximal end side of the operation part and that is rotatable relative to the operation part in the direction around the axis; and
an extension part that is provided in the extending part and that connects the extending part to the second bearing receiving member.

19. The endoscope according to claim 1,
wherein the shaft member is an inner sheath,
the endoscope further comprises a proximal end optical system that is provided at a distal end of the inner sheath and that guides light, which passes through the distal end optical system, to the image pickup unit,
the image pickup unit includes an image pickup element that picks up an image of light incident through the proximal end optical system, and
the proximal end optical system includes a proximal end lens barrel that is fixed to the distal end of the inner sheath, a first refractive optical element that is connected to the image pickup element and that refracts light, which is incident from the proximal end optical system, toward the image pickup element, and a holder that holds the first refractive optical element on a proximal end side of the proximal end lens barrel.

20. The endoscope according to claim 1,
wherein the distal end optical system includes a second refractive optical element that refracts light, which is incident in a direction inclined with respect to the insertion axis, parallel to the insertion axis.

21. The endoscope according to claim 20,
wherein the distal end optical system includes a distal end portion body and a distal end lens barrel fixed to the distal end portion body, and
the distal end lens barrel houses the second refractive optical element.
